(19) United States
(12) Patent Application Publication
(10) Pub. No.: US 2026/0348821 A1
(43) Pub. Date: Nov. 5, 2026

(54) METHOD FOR DETERMINING A SUBSTANCE CONCENTRATION AND DETECTOR DEVICE

(71) Applicant: ams-OSRAM AG, Premstaetten (AT)

(72) Inventor: Klaus FLOCK, Regensburg (DE)

(21) Appl. No.: 19/487,329

(22) PCT Filed: May 23, 2024
(86) PCT No.: PCT/EP2024/064311

§ 371 Date: Nov. 24, 2025

(30) Foreign Application Priority Data
May 24, 2023 (DE) 10 2023 113 575.9

(51) Int. Cl. G01N 15/075; G01N 15/01

(52) U.S. Cl. CPC G01N 15/075; G01N 15/01; G01N 2015/012

(57) ABSTRACT

A method for determining a substance concentration in a sample comprising liquid containing particles, in particular oxygen saturation includes the steps of obtaining a first perfusion index based on a reflected first component of an incident first light beam with a first wavelength, obtaining a second perfusion index based on a on a reflected second component of an incident second light beam with a second wavelength, obtaining a reflected third component of an incident third light beam with a third wavelength, wherein the third light beam is in a wavelength range that is less sensitive to oxygen saturation than the first and second light beams, adjusting at least one of the perfusion indices in response to the obtained third component, and determining the oxygen saturation or a change thereof from the at least one adjusted perfusion index and the respective other perfusion index.

The present application claims priority from DE patent application 10 2023 113 575.9 dated May 24, 2023, the disclosure of which is incorporated herein by reference in its entirety. The present invention relates to a method for determining a substance concentration in sample containing particles in a liquid, in particular glucose in blood, wherein a refractive index of the liquid is dependent on a concentration of the substance dissolved therein. The invention also relates to a detector device.

### BACKGROUND

The current standard for blood glucose measurement often uses an invasive technique in which a small amount of blood is drawn, and subsequent electrochemical analysis is performed using a handheld device. This method is not suitable for continuous monitoring because for each measurement, the finger must be pricked to obtain a fresh blood sample.

A more recently developed technology uses a button that sits on the skin and misses interstitial fluid in parts of the subcutaneous adipose tissue with a small, needle-like sensor. However, the needle penetrates permanently into the skin.

Besides these invasive methods, there are also non-invasive methods based on optical IR measurements or Raman spectroscopy. While in the first case, a suitable choice of emitter and detector leads to difficulties, an approach based on Raman spectroscopy is challenging due to the very poor signal-to-noise ratio.

In this respect, there is a need for a method that can detect a substance in a liquid in a simpler way and allows for a continuous measurement.

### SUMMARY OF THE INVENTION

This and other objects are addressed by the subject matter of the independent claims. Features and further aspects of the proposed principles are outlined in the dependent claims.

Complementing some other ideas that are based on an angle dependent scattering, the present idea relies on the evaluation of the modulation depth, also referred to as perfusion index, which has been found by the inventors to also depend on the blood glucose concentration.

The perfusion index or modulation depth is a result during so-called Photoplethysmography or PPG, i.e., the optical measurement of volume changes of human blood, e.g., due to the beating of the heart, at a certain point of the human body, for instance the fingertip, or some other suitable location. The volume changes are observable by signal variations during the heartbeats resulting from less or more scattering based on the different blood volume.

Due to scattering and absorption of light, which is initially directed towards the skin and may subsequently propagate through portions of shallow-and/or deeper lying tissue, which in turn hosts a network of blood vessels, the amount of light that re-emerges at the skin-to-ambient interface some distance from the entry point will vary according to the optical path length (distance) travelled in blood. The path length is referred to as “blood optical path length (BOPL),” which in turn is directly affected by the beating of the heart. Consequently, the BOPL varies with time, while the properties of surrounding portions of tissue can be considered constant, at least on the timescale of the heart rate.

When the BOPL is at a maximum or minimum, the emerging signal after interaction is conversely at a minimum or maximum. The difference between minimum and maximum signal is called modulation, and the ratio of modulation to average signal is defined as modulation depth, ac-to-dc ratio, or perfusion index.

According to Beer-Lambert's law, the absorption of light in blood is largely determined by the presence of Hemoglobin within the erythrocytes, as well as the cumulative BOPL. Under the assumption that the density of erythrocytes does not change during the measurement period, one can say that light propagating nominally through the more or less transparent blood plasma, undergoes scattering at the red blood cells, such that the cumulative BOPL from entry point to detection point, both of which are fixed by the device configuration, may depend on the scattering characteristics in the sense of a random walk, i.e., it will become a stochastic process.

Consequently, the perfusion index or modulation depth is primarily a result of the direct interaction and particularly the scattering between light and erythrocytes.

This principle is currently used for measurements of the oxygen saturation in blood, also referred as SPO2 measurement. SPO2 measurements are based on the ratio of PPG signals recorded at two different wavelength, i.e. in the red and IR spectrum. More specifically, the SPO2 measurement if performed by generating the ratio of perfusion indices at the two different wavelengths. This is accomplished with a setup, where a red and an IR emitter for example are used to illuminate tissue, e.g., by alternately transmitting short pulses of light at a sufficiently high repetition rate, e.g., 20 Hz, 50 Hz, 200 Hz, etc., and sampling the corresponding photocurrent generated in the photodiode. The raw photodiode current consists of an average signal level, referred to as DC, and a modulation of the signal due to the beating of the heart, referred to as AC. The AC signal can be estimated, for instance, by the difference between the peaks and the valleys of the raw signal. The ratio of AC to DC is called the perfusion index (PI) or relative modulation or modulation-percent. The reference “In Vivo Reflectance of Blood and Tissue as a Function of light wavelength”, Weijia Cui et al. IEEE Transactions on biomedical engineering, Vol 17, no 6. June 1990.

The ratio of the relative modulations is computed to estimate the oxygen saturation. This process is also called the ratio of ratios. By proper calibration of such values with a reference device one can identify early 100% of oxygen saturation and lower levels thereof.

However the ratio of ratios for a given oxygen saturation and thus an SPO2 measurement depends on optical design choices, in particular the relative separation between the light source and detector for each wavelength, the emission characteristics of each light source, the detection characteristics of the photo diode, and the performance of the optical barrier in between sources and detectors, as well as other system aspects that affects how light may propagate from a source to a detector. Still, the above-mentioned approach to optical SPO2 measurements is widely discussed in the literature and scientifically accepted.

It has now been observed that for a human test subject under steady-state conditions and constant blood glucose levels, the perfusion index remains substantially constant. When the blood glucose level increases, the properties of the blood plasma and more particularly the refractive index of the plasma changes. As a result, the difference between the refractive indices of the erythrocytes and the blood plasma decreases, which will cause a decreases of scattering light. Likewise, a decrease in the glucose increases the difference between the refractive indices of the erythrocytes and the blood plasma causes an increase in scattering. This is explained in a model, in which scattering is mainly caused by diffraction differences, thus in a transparent medium, in which the refractive indices of the liquid and the scattering particles were the same, no scattering would be observable, since light would simply propagate through the medium without interaction. However, once there is an index mismatch, some light is being scattered (e.g., backwards) by the particles and thus can be distinguished from the surrounding ambient, i.e., plasma.

Consequently, one can use the perfusion index for so-called Oral glucose Tolerance Test, in which a test subject starts with a baseline blood glucose level established by not eating for several hours followed by ingesting a liquid or substance of caloric value. Usually, changes in the perfusion index become visible after around 5 to 15 minutes after the intake. The change of perfusion index indicating a change in the glucose level may inherit some benefits, as one can use a steady state as a reference value, such that a single measurement of the perfusion index allows to derive a blood glucose change therefrom. It has further been found that the changes in the perfusion index are also dependent on the wavelength at which the perfusion index is measured. In other words, similar to the SPO2 measurement the ratio of ratios is also changing for different wavelength. This observation has significant impact for the SPO2 measurements.

Particularly, it means that some 10 minutes after glucose intake, the SPO2 measurement based on this bench-top configuration may indicate an oxygen level that is different from the actual oxygen saturation level. As the glucose level directly impacts the SPO2 measurements, the latter can be improved by taking the glucose level into account or at least compensate for the portion in the SPO2 measurements that may be caused by the glucose level change over time.

The inventor proposes several different approaches thereto. In some aspects, a method for determining the oxygen saturation in blood is proposed. The proposed method is based on the above-mentioned perfusion determining measurements but adjusted to compensate for a possible variation caused by the glucose intake. For this purpose, a first perfusion index based on a reflected first component of an incident first light beam with a first wavelength is obtained. Likewise a second perfusion index based on a on a reflected second component of an incident second light beam with a second wavelength is obtained.

Both components can for example be obtained by emitting the respective light beam onto a human tissue and subsequently collecting a reflected light portion. The emission of the respective light beams can be periodic, for example in form of a plurality of light pulses, e.g. with a frequency of larger than 10 Hz and for example in the range between 20 Hz and 100 Hz. The high frequencies are substantially faster than the heart beat allowing to determine a plurality of sample representing the heartbeat.

The plurality of signals caused by the detection of the reflected components at the different wavelength of light are used to determine respective wavelength dependent perfusion indices. In accordance with the proposed principle the perfusions indices, or the obtained detected light components are adjusted to compensate for glucose intake or other substances.

This is achieved by further obtaining a reflected third component of an incident third light beam with a third wavelength, wherein the third light beam is in a wavelength range that is less sensitive to oxygen saturation than the first and second light beams.

This step makes use of the fact, that light of certain wavelengths is substantially insensitive in regards to blood oxygen saturation but are otherwise strongly correlate with light of other wavelength in regards to another substance. Consequently this correlation is used to adjusting at least one of the first and second perfusion indices in response to the obtained third component or a value derived therefrom.

The oxygen saturation or a change thereof from the at least one wavelength is then determined using the one adjusted perfusion index and the respective other perfusion index. In an alternative solution, the oxygen saturation or a change thereof from the at least one wavelength is determined based on the perfusion indices of the first and the second components as well as the obtained reflected third component. The use of the third component is particularly beneficial as it breaks the correlation of the first and second light beam in regards to the blood saturation and the other substance like glucose that influences the measurement of blood Oxygen (?) saturation.

In some further aspects, one can further improve the results by emitting at least one of the light beams and particularly the first and second light beam or detecting the respective light beams in a range close to the normal incidence, that is within a range between 0° and less than 45°, so for example less than 35° or 30°. The improvement is fuelled by the fact that signal levels for higher angles (to the normal incidence) outweigh the ones for the lower angles. Hence, higher angles of incidence and detection exhibit a different correlation to glucose concentrations and thus could be particularly damaging for SPO″ measurements/applications.

More specifically, it has been observed that, for higher angles of illumination and detection, the response of the perfusion index to blood Glucose concentration changes can be more pronounced for red wavelengths than for IR wavelengths such that the ratio of ratios retains a Glucose signature. This signature is by-and-large absent from the ratio of ratios when the angles of illumination and detection are limited to near normal incidence.

The above principle is not limited to SPO2 or oxygen saturation measurement. Rather, the observation leads to the conclusion, that a refractive index of the liquid is dependent on a concentration not only of the substance dissolved therein (the one to be determined) but also by another dissolved component as well. In other words, the refractive index change is caused by concentration variations of the substance and the components. Depending on the combination, the variations of the refractive indices may compensate each other but also add up, such the overall result is heavily skewed.

Hence, the inventor proposes a method that more generally determines a substance concentration in a sample comprising liquid containing particles, wherein a refractive index of the liquid is dependent on a concentration of the substance dissolved therein and a density of particles in the liquid is substantially constant.

In a first step thereof a first light beam with a first wavelength is emitted onto the sample containing the liquid and a first light component of the first light beam scattered by the sample is detected. Likewise, a second light beam with a second wavelength is emitted onto the sample containing the liquid and a second light component of the second light beam scattered by the sample is detected.

A ratio of the perfusion indices of the first light component and the second light component is then determined, whereas the determination is adjusted to compensate for a possible skew of another substance. The adjustment is achieved by one or more steps. For example, one may adjust the perfusion indices of at least one of the first light component and the second light component by a factor derived from a third light component of a third light beam with a third wavelength scattered by the sample in response to the third light beam emitted onto the sample.

Alternatively, a perfusion index can be obtained based on a third light component of a third light beam with a third wavelength scattered by the sample in response to the third light beam emitted onto the sample. This perfusion index is then used to adjust the perfusion indices of the first and second scattered light components.

Alternatively or in addition, at least one of the first and second light beam can be emitted in the range between 0° and 45° and in particular between 0° and 35° and in particular less than 30° of the normal incidence. Likewise, at least one of the first and second light component can be detected in the range between 0° and 45° and in particular between 0° and 35° and in particular less than 30° of the normal incidence. In some aspects, the overall angle between emission and detection should be smaller than 60° and particularly smaller than 45° in regards to the normal incidence.

The substance concentration or a change in the substance concentration is then derived from the adjusted perfusion indices.

In some further aspects, the inventor proposes to use the ratio of ratios that is the ratios of two perfusions beams at two different wavelengths to obtain the glucose concentration in a liquid containing particles, in particular blood. Consequently, in some aspects, one may emit two light beams at different wavelength and obtain the respective components scattered within the sample therefrom. The two components are then processed to determine the respective perfusion indices thereof, and the glucose concentration is derivable from the ratio of those ratios.

In some aspects, the liquid contains at least two dissolved substances, one being the substance to be measured, for example the oxygen saturation, the other one being an undesired substance. The latter influences-as mentioned above-the determination of the substance concentration to be measured. In particular the liquid containing particles comprises a component in some instances, in particular glucose that influences the refractive index of the liquid. In some aspects, scattering the third light beam with the third wavelength is substantially insensitive to a change of concentration of the substance dissolved in the liquid. Hence, there might be a correlation between the third light beam and the concentration of the component not to be determined, but there is substantially no or only a very small correlation between the concentration of the substance and the third light beam.

Further in some instances, the scattering of the first light beam, the second light beam and the third light beam is dependent on a concentration of another substance dissolved in the liquid and particularly of a component which influences the measurements of the substance concentration.

Some aspects concern the detection and emission. In some instances, the first component, the second first component and optionally the third first component are detected from substantially the same location on the sample. In some more aspects, the location of the first, second and optional third light beam that is incident onto the sample may be different. In some aspects, the angle of the incident light of one of the light beams or the angle of the reflected light of one of the components to the normal incidence may be different than the angle of the respective other light beams and/or components. However, it may be advisable in some instances, if the angle of the incident light beam and/or the angle of the respective reflected component with the longer wavelength is also smaller, that is closer to the normal incidence. This is beneficial as it has been found that light beam with larger wavelength show a stronger dependencies towards glucose and other substances in blood, which can screw the blood saturation measurement.

In some aspects, the third wavelength is in the range between 500 nm to 550 nm. Those wavelengths are in the green range of the visible spectrum and light with such wavelength is not very sensitive to the concentration or saturation level of oxygen in blood. However, light with such wavelength correlates with light in other regions of the spectrum in regards to other substances like glucose. Consequently, light in the green range can be used to compensate when calculating the perfusion indices of light being sensitive to the blood saturation. For example, the correlation between green light and glucose can be used to subtract this level from one of the other light amplitudes. This principle can of course be generalized and used in all measurement, in which the perfusion index of light of different wavelengths for a given substance concentration is skewed by another component.

In some aspects, the first light beam is light beam is in the range between 580 nm and 650 nm and thus in the red spectrum. The second light beam may be in the infrared range with a wavelength larger than 670 nm. These two spectra are suitable for human tissue as the light is able to penetrate into deeper portions below the skin.

It has nonetheless surprisingly found that it is not necessary to conduct two measurements to obtain the concentration or the concentration change in a liquid. This applies not only for the above-mentioned glucose measurements, but also for the SPO″ measurement.

Rather, one can define a reference value and then conduct a single measurement. Using the reference, one is able to derive the substance concentration or changes thereof. Consequently, the inventor proposes a method comprising the steps of obtaining a reference value associated with or corresponding to at least one of a scattered light components, or a signal derived therefrom.

For example, one of the measurements is then replaced by the reference value, which can be obtained by several means prior to the actual measurement. In this regard, it is understood that the reference value may comprise not only the intensity signal but can also refer to any value, from which the reference concentration of a substance or a signal to be processed subsequently can be meaningful derived.

Consequently, the term reference value shall be understood as being equivalent to any value from which the substance concentration or the intensity signal can be derived. In accordance with the proposed method, the substance concentration or a change in the substance concentration is derived from the changes between the reference value and the respective other intensity value or a value derived therefrom.

The reference value and also characteristics from a curve corresponding to the change of the intensity signal in relation to the substance concentration, the SPO2 level or the blood glucose level are dependent on various factors. Hence, one may utilize those factors, when obtaining the substance concentration. These include, but are not limited to the age, size, weight, gender, body mass index and skin type. Particularly, skin type can be used not only to adjust the processing of the respective intensity signals, but also adjust the wavelength of the emitted signals.

In some aspects, the reference value is a statistical value obtained as a general normalized intensity signal (different from zero) from a plurality of different previous measurement. Those may take one or more of the above parameters into account to obtain a small range with certain confidence level i.e. 2σ or 3σ.

Some other aspects concern an optoelectronic device for determining a substance concentration in a sample comprising a liquid containing particles, in particular the oxygen saturation, although many other substances are suitable as well. The optoelectronic device comprises a housing with an exit window and an optional entrance window as well as at least one emitter unit arranged below the exit window. The at least one emitter is configured to emit a first light beam during and a second light beam from the exit window onto the sample.

The optoelectronic device further comprises at least one photodetector arranged below the entrance window. The at least one photodetector is configured to detect a first component scattered by the sample in response to the first light beam and a second component scattered by the sample in response to the second light beam. An evaluation unit is coupled to the at least one photodetector. It is configured to receive signals from the photodetector response of the first and second components. The evaluation unit determines a modulation depth of the respective first and second components; and subsequently determines the ratio of those two modulation depths. The evaluation unit is configured to determine the oxygen concentration or a change thereof or more generally a change in a substance concentration in the sample from the changes between the first and the second modulation depths.

In this regard, the modulation depth or the perfusion index PI is characterised by a ratio of an AC portion and a DC portion of the respective detected component. The AC portion thereof corresponds to a varying component part of the respective detected components and the DC portion corresponds to a substantially constant component part of the respective detected components. By evaluating these components and calculating the ratio thereof, one can derive information about a change in the substance concentration in the sample.

As an alternative to an evaluation using the two or more scattered components or the modulation depth, one may utilize a reference value. Hence, only a single measurement is required to determine a substance concentration or a change in the substance concentration in the sample. In such alternative, the control unit is configured with a memory storing one or more reference values. The control unit is further configured to determine a substance concentration or a change in the substance concentration in the sample utilizing the reference value and one of the first and second scattered components, the modulation depth or the ratio thereof or values derived therefrom.

Said reference value is associated with or can correspond to-as previously mentioned above-different characteristics including, but not limited to scattered component, a modulation depth or a signal derived therefrom. It can correspond to a normalized level, which may be individual or obtained from statistical value or derived over a longer period of time, e.g. a long measurement time of the respective user. Furthermore, the reference value is associated with or can correspond to a function that links one of the above-mentioned parameters with the perfusion index or a ration of the two scattered components, with the modulation depth or any other signal characteristics. Likewise, such function is equivalent to a function that links the above-mentioned signals to glucose level, to the perfusion index or any other signal characteristics.

The reference value can be derived as mentioned above, i.e. individually for each person or from a plurality of measurements as a statistical normalized value or function. This can take at least one of body mass index, age, gender, height and weight and skin type into account.

In some aspects, the emitter unit is further configured to emit from the exit window onto the sample a third light beam having a third wavelength, said third wavelength selected such that the third light beam is scattered by the sample substantially independent of the substance concentration. The at least one photodetector arranged below the entrance window may be adapted to detect a third component scattered by the sample in response to the third light beam.

Consequently, the evaluation unit is then configured to adjust at least one of the modulation depths and/or the ration of both modulation depth in response to the detected third component. In other words, the additional third component is used by the evaluation unit to compensate for a dependency of the first or second component to second substance in the sample. The third light beam may be in the green range of the spectrum, particularly between 510 nm and 560 nm or the value specified herein.

In some other instances, at least one of the emitting angle of one of the first, second and third light beam and the detecting angle of the first, second and third component is in the range between 0° and 35° in regards to the normal incidence of the sample. This aspect addresses the dependency between the angle of incident or detected light and the respective wavelength. By limiting the angle to small values in regards to the normal incidence, one can reduce this dependency.

On some aspects, the optoelectronic device further comprises an optical barrier arranged in the housing and extending from the exit window towards the housing bottom arranged between the emitter unit and the photodetector unit. This will reduce the stray light and improve signal/noise ratio. In addition several structures can be arranged on the detector or the emitter to limit the radiation and detection characteristics, thereby deviation from a usual Lambertian one. For example these structures may include a DBR or Bragg grating structure arranged on the at least one photodetector. Similar, an aperture can be arranged on the at least one photodetector. One or more collimating lenses can be placed on at last one of the emitter unit and the at least one photodetector. All of these aspects can be combined. In addition, one may change the type of emitter, using for instance VCSELs, which already comprise a more limited radiation characteristic.

To obtain a certain symmetry and compensate for irregularities on the sample surface, the emitter unit can comprise a ring-shaped form arranged around the at least one photodetector in some aspects. Alternatively or in addition the at least one emitter unit is arranged around the at least one photodetector. Location of photodetector and emitter in this regard can be exchanged if needed.

In some instances, the evaluation unit is configured to perform an FFT on the detected first and second components to obtain a frequency spectrum thereof. Any kind of analogue or digital low-pass or bandpass filter (if the component is converted to the digital domain) can be applied on the detected components prior to performing the FFT or on the frequency spectrum (that is after performing the FFT) . The optional low-pass or band-pass filter comprises a cut-off frequency below 100 Hz and in particular below 50 Hz. The fundamental peak in the respective spectra and/or the harmonics thereof are obtained to derive information about the concentration substance.

In some alternative aspects, the evaluation unit is further configured to obtain a moving average from the detected first and/or second components; and subsequently subtract the obtained moving average from the respective detected components prior to performing an FFT. This will reduce outliers or other errors not caused by the scattering itself.

In some further alternatives, the evaluation unit is configured to normalize the respective detected first and second components. Then, a maximum peak of the normalized component in the respective duration. Also a minimum adjacent to the respective maximum peak, either prior to the peak or following it is identified. The amplitude between the minimum and the maximum can be obtained therefrom.

In some aspects, the optoelectronic device contains an emitter unit capable of producing light of different wavelength, including, but not limited to red, green, and infrared. During measurements those lights are emitted sequentially. Alternatively, the detector unit also comprises several detectors having respective filter attached to it such that they pass light of dedicated wavelength. In this regard, any filter on the photodetector unit may be suitable to block ambient light.

Some other aspects concern the implementation of the optoelectronic device according to the proposed principle. In some aspects, the optoelectronic device also comprises an optical barrier arranged in the housing and extending from the exit window towards the housing bottom arranged between the emitter unit and the photodetector unit. Such barrier will prevent light from the emitting light source to directly reach the respective photo detectors.

In some further aspects, the emitter unit and/or the photodetector unit comprises an optical system, which is designed to generate a focal point on the sample. This will maximize the light on the sample and improve the signal to noise ratio. In some aspects, a focal point of the incident light, which is light generated by the emitter unit is spaced apart from a point from which outgoing light at a first angle is detectable by the photodetector unit. This will reduce cross talk during the measurement.

Although the optoelectronic device presented herein is only illustrated with regards to its functionality concerning the determination of substance concentration, one may note that various implementations are possible. In this respect, the control unit, evaluation unit or portions thereof does not need to be implemented within the housing itself containing the emitter and the detector but can be located separately therefrom. In some aspects, the control and/or evaluation unit is implemented in a separate device distances from the housing itself. Communication between the emitter and detectors and the control and/or evaluation unit as described above is facilitated for example by a wireless communication. This will allow for example to realize a master slave configuration, in which the control unit request measurement to be taken on regular basis. Furthermore, the control and/or evaluation unit may be implemented largely in software, for example as an app executed on a mobile device, whereas the remaining portion of the detector device are implemented in a separate housing.

In some aspects, the housing (or the optoelectronic device in more general terms) is implemented as a ring, earbud, watch or any other wearable, that may fit in some aspects into a user's usual environment and can be carried continuously. Said ring, earbud, watch or any other wearable is in communicative connection with the control unit or a mobile or any other device implementing the control unit. The ring, earbud, watch or any other wearable may cover a larger portion of the user's skin, e.g. wrap around the finger, clipped to the ear from both sides and the like. They may contain several emitters and detectors at various location, thus allowing not only to measure at one spot but at several at once or subsequently. As a result thereof, skin irregularity or other issue can be overcome and the overall measurement quality improved.

Apart from devices and handheld devices, other applications are possible. For example, the optoelectronic device can be implemented in medical devices or laboratory equipment, e.g. for test and measurement purposes. Those devices again can be stationary or mobile.

Some more aspects concern mobile displays in which the optoelectronic devices of the proposed principle are directly implemented. In such applications, the display LEDs e.g. for the red and green colour can be used as emitter in accordance with the proposed principle. A finger is placed directly on the display surface and then illuminated by the display for obtaining the first and/or second signal. Likewise, the proposed principle can be implemented in VR or AR glasses and devices. Another application concerns safety issued, e.g. during certain labour work but also during driving and the like. It is possible to implement such detector devices in accordance with the proposed principle in a car, e.g. on the steering wheel to obtain the intensity signals and the changes thereof during driving. This enables for example warnings to the driver of potential health threats while driving.

### SHORT DESCRIPTION OF THE DRAWINGS

### Further Aspects and Embodiments in Accordance With the Proposed

principle will become apparent in relation to the various embodiments and examples described in detail in connection with the accompanying drawings in which

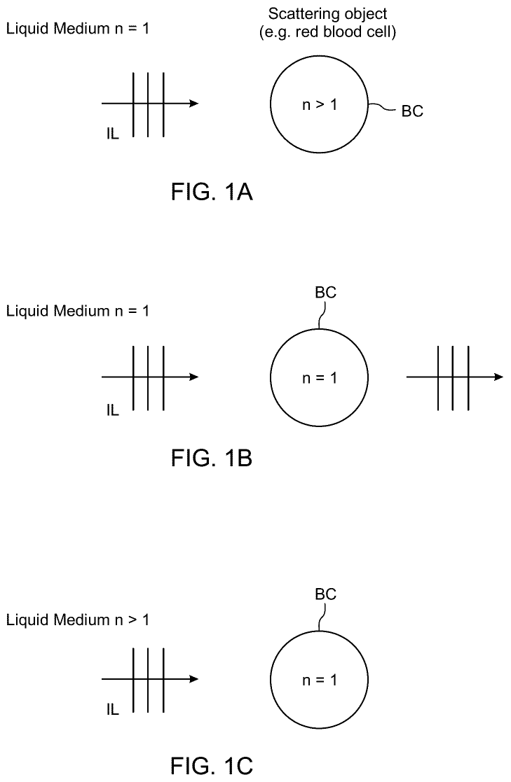

FIGS. 1A-1C show a simplified model a scattering mechanism in a particle containing liquid containing;

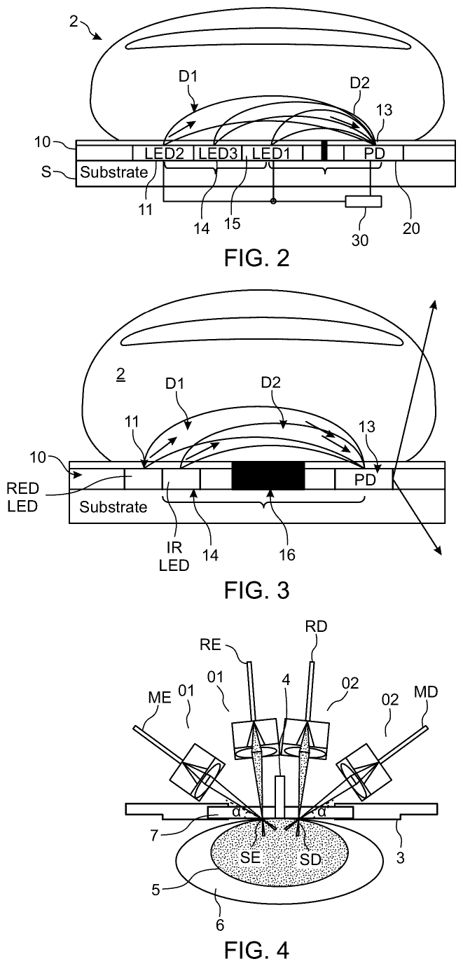

FIG. 2 shows a first embodiment of a sensor arrangement in accordance with some aspects of the proposed principle;

FIG. 3 shows a second embodiment of a sensor arrangement in accordance with some aspects of the proposed principle;

FIG. 4 illustrates a test setup of a sensor arrangement in accordance with some aspects of the proposed principle;

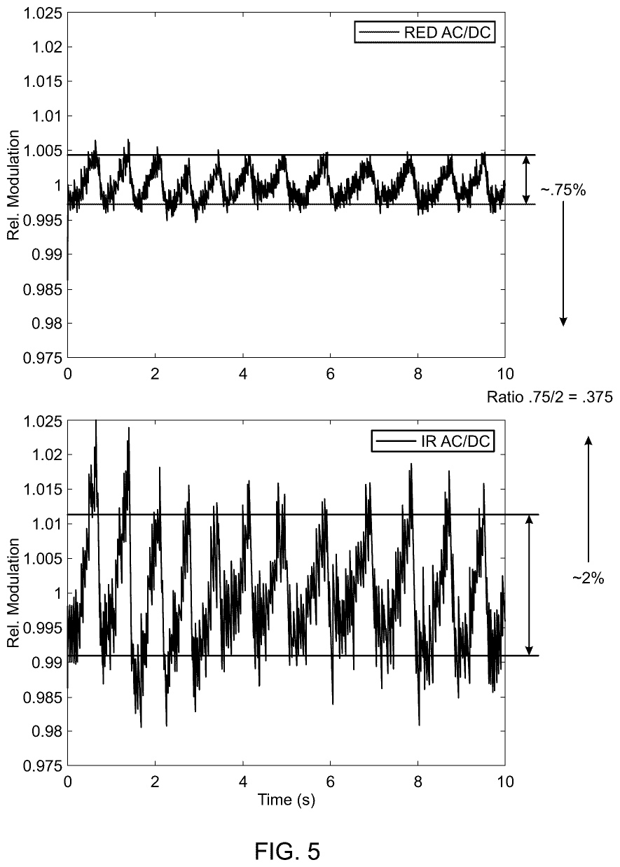

FIG. 5 shows the perfusion indices obtained at red and infrared light respectively for an SPO2 determination;

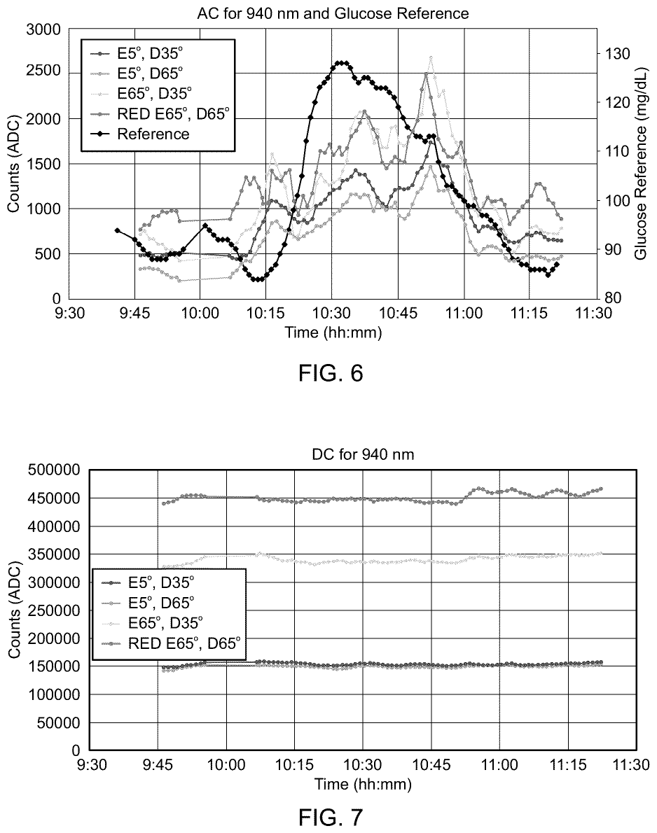

FIG. 6 illustrates a diagram for the AC values obtained at a wavelength of 940 nm for a plurality of different angles;

FIG. 7 shows a diagram for the DC values obtained at a wavelength of 940 nm for a plurality of different angles;

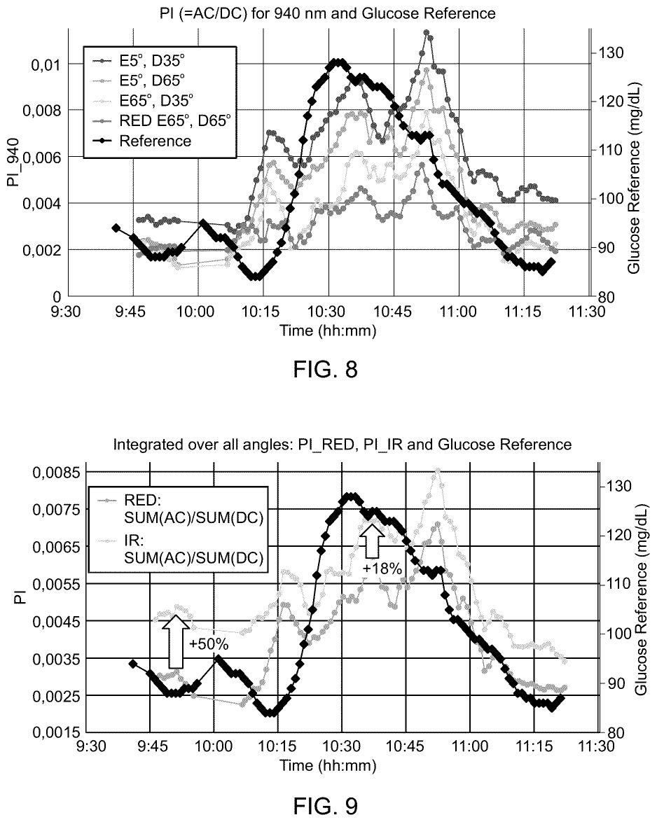

FIG. 8 illustrates the corresponding perfusion index for the respective values obtained previously;

FIG. 9 illustrates the sum over all angular measurement to illustrate some dependencies pointing to some aspects of the proposed principle;

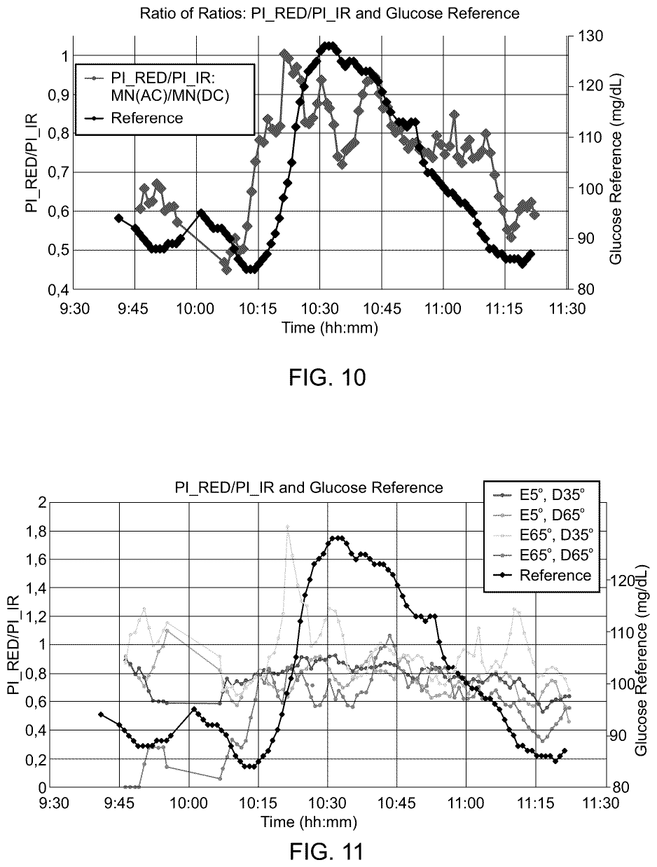

FIG. 10 shows the ratio of perfusion indices and a reference measurement to outline some aspects of the proposed principle;

FIG. 11 illustrates the ratio of perfusion indices in dependence of the angles of incident and detected light;

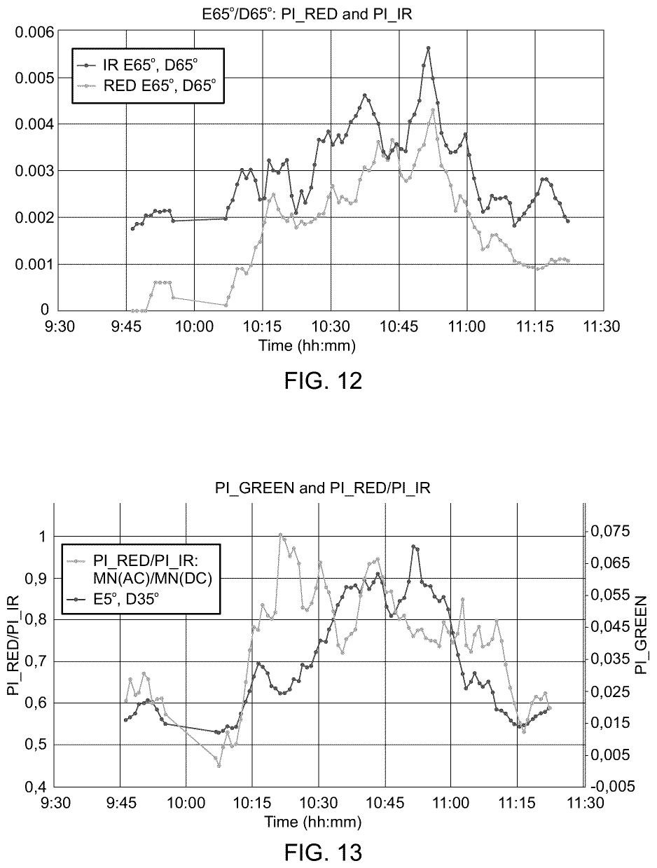

FIG. 12 shows the different perfusion indices for red and infrared light at large incident and detected light;

FIG. 13 shows the correlation between light of the green spectrum and ratio of perfusion indices suitable for compensation of glucose impact during SPO2 measurements.

### DETAILED DESCRIPTION

The following embodiments and examples disclose various aspects and their combinations according to the proposed principle. The embodiments and examples are not always to scale. Likewise, different elements can be displayed enlarged or reduced in size to emphasize individual aspects. It goes without saying that the individual aspects of the embodiments and examples shown in the figures can be combined with each other without further ado, without this contradicting the principle according to the invention. Some aspects show a regular structure or form. It should be noted that in practice slight differences and deviations from the ideal form may occur without, however, contradicting the inventive idea.

In addition, the individual figures and aspects are not necessarily shown in the correct size, nor do the proportions between individual elements have to be essentially correct. Some aspects are highlighted by showing them enlarged. However, terms such as “above”, “over”, “below”, “under” “larger”, “smaller” and the like are correctly represented with regard to the elements in the figures. So it is possible to deduce such relations between the elements based on the figures.

FIGS. 1A to 1C illustrate a model for the conceptual propagation of light in a liquid and heterogonous medium. The medium comprises a refractive index of n=1 as illustrated in FIG. 1A. The incident light IL propagating through the liquid medium is scattered on a solid object BC within the liquid. The object can be a blood cell, for instance in case of blood plasma as liquid medium. However, the model as well as the proposed principle explained later on is not limited to blood plasma, but also encompasses any other scattering object having a refractive index of a larger than 1 in a liquid medium.

In the case of FIG. 1A with n=1 for the liquid medium and the scattering body having n>1, the incident light IL will partially be reflected at scattering body BS but also scatter thereupon with large portion in the forward direction as indicated in the Figure. The amount of back scattering as well as forward scattering is based on the difference between the refractive indices in the liquid medium as well as on the scattering object.

In FIG. 1B, the refractive index of the liquid medium is the same, that is n=1, while the scattering object now comprises a different refractive index equal to the refractive index of the medium (e.g. n=1 as well). According to the conceptual propagation, this means that the incident light will not see any refractive index deviation between the scattering object and the liquid medium. As a result, the incident light will propagate through the medium and not be scattered by the object BC.

For FIG. 1C, the liquid medium comprises a refractive index of larger one, n>1. Here the refractive index of the scattering object is equal to 1, but more importantly the refractive index of the scattering object is smaller than the refractive index of the surrounding medium. Consequently, the situation is similar to the one depicted in FIG. 1A with the incident light being partially reflected backwards and partially scattered on the object BC. The difference between the schedule back scattering and forward scattering is again based on the differences between the respective refractive indices.

In accordance with the model, the ratio of back scattering to forward scattering will change when the difference between the refractive indices changes. Such variation the refractive indices and the difference between both indices occur when the concentration of a substance dissolved in the liquid medium changes. However, one may also observe a change of the refractive index depending on the particles characteristic themselves. This is the case of oxygen measurements, in which oxygen is bound to the red blood cells. The blood cells having oxygen bound to it behave differently and result in a slightly different scattering compared to red blood cells with no oxygen or carbon dioxide bound to it. The dependence on the refractive index changes therefore depends on the amount of oxygen bound to the red blood cells, referred to as O2 saturation, but also on the wavelength of the incident light.

In case of blood plasma, such substance can be glucose for example, but can also include other components or substances. Often several substances have a certain influence on such changes, and even more the influence of the substance concentration may also be dependent on the wavelength of the incident light.

Hence, the refractive index of blood plasma varies with varying concentration of oxygen and glucose dissolved in it and can consequently be used for evaluation of the overall absolute and/or relative concentration. However du to its similar behaviour, one need to know the concentration of the respective other substance to compensate for the effect, which also caused by different processes result in refractive index change.

It is assumed for a measurement of the oxygen and/or glucose concentration in blood, that the propagation of light from the light source to a light detector through tissue of a body and blood plasma will result in scattering and absorption of light, whereas the light takes a random path through the tissue. Depending on the scattering characteristics of the tissue, the path taken from the light source to the detector could be different. Particularly, scattering and absorption in the tissue are dependent from wavelength with generally a higher absorption at shorter wavelength. Further, the amount of scattering is dependent on the distance between the light source and the detector when the resulting index contrast-that is the difference of the refractive indices between the blood plasma as well as the red blood cells-is modulated depending on the oxygen and/or glucose concentration.

For such measurement several implementations of detector devices can be used. FIG. 2 and FIG. 3 each illustrate an exemplary embodiment for such measurement devices suitable not only to detect and derive a substance concentration in blood, like oxygen or glucose, but also for a variety of further measurements.

The detector device 1 in FIG. 2 comprises a substrate S, which is embedded in a housing 10 or forms a part of it. Housing 10 also comprises three light emitting devices 11, 14 and 15 in form of an optoelectronic component or a lighting diode, LED. The lighting diodes are configured to emit light of a specific wavelength through an exit window 12 on the top surface of the housing 10. Diode 11 for example emits green light or light within the green spectrum, diode 14 emits red light and diode 15 emits light in the infrared spectrum. Although the diodes 11, 14 and 15 are illustrated herein to be spaced apart, one should note that they are actually close together or centred around the location of photodiode 13, such that its distance d to the photodiode 13 is approximately the same. The latter ensures that light emitted by the respective LEDs scattered through the tissue will eventually reach the photodiode under the same angle.

A barrier 16 is arranged between the respective LEDs and the photodiode to suppress any light from the diodes to reach the photodiode directly and not scattered through the tissue.

An entry window with a respective filter element is applied in front of the respective photo diode 13. The filter element is transparent for the wavelengths of light emitting diode 11, but otherwise comprises a high absorption or reflection for wavelength outside that the respective frequency band. The entry windows will reduce the amount of ambient light being detected by the respective photo diode 13 during the measurement cycle. In an alternative embodiment or also additionally, the photo diode 13 may be sensitive to the wavelength, but otherwise insensitive. Housing 10 further comprises a control and evaluation circuit 30, which is connected to the light emitting component and related to directing components, respectively.

FIG. 3 illustrates a similar embodiment of a detector device in accordance with the proposed principle. However, the detector device illustrated in FIG. 3 shows a first light emitting component 11 and a second light emitting component 14, which are directly adjacent to each other. It may include a single device with a broader emission spectrum covered by two or more respective wavelength filters. Similar to some other embodiments, the light emitting components can form a ring shape structure cantered around the photodiode. Blocking barrier 16 prevents direct light from the diodes 11 and 14 to reach the photo diode. In addition, the surface of photodiode 13 is covered by a DBR structure that prevents light with an incident angle smaller than a threshold from a normal incidence from reaching the photo diode's surface. Consequently only scattered light witch is substantial perpendicular (or has only a small deviation therefrom) reaches the photodiode 13.

The optical path D1, D2 length illustrated in the FIGS. 2 and 3 is given by the light traveling from the spot on the sample's surface for the incident light to the location at which exiting light is detected. For a good measurement, the shape and structure of the top surface of the housing that is in the particular the exit window in front of the light emitting components 11 and 14 and the entry window in front of the detecting component 13 should be shape such that it is substantially light tight when a sample is placed upon the respective windows. In case, in which the sample is compressible, the sample should be a slightly pressed on the respective windows to further reduce any incident or ambient light from reaching the light detecting components. Likewise, the exit window is tightly arranged against the sample to prevent ambient light from entering the incident location at the entry sample surface.

It is suitable if the difference between the photodiode 13 and the light emitters 11 and 14 are substantially equal to avoid possible effects due to different optical path length through the tissue. Alternatively, a different length can be taken into account when the respective rations for the perfusion indices are calculated.

It should be noted that the positions of the photodiode and the light emitting diodes can be replaced, and more than a single photodiode can be positions on the sensor. Due to the fact that light intensity ratios and not absolute light strengths are evaluated in accordance with the proposed principle, it is sufficient to consider the known and measurable distances between the light emitting diode and the photodiode, which receives said light from the light emitting diode.

FIG. 4 shows a test arrangement to illustrate various aspects of the proposed principle, among those the dependency of the perfusion index onto the incident angle. The test arrangement 1A shown in FIG. 1 is placed on the skin surface of a person to be diagnosed. The essential components of the detector arrangement are listed here again by way of example. The skin surface is marked with the reference sign 6 and is, for example, a part of the finger or another skin area. For this purpose, the test arrangement comprises a frame 3 with a window 7, which is placed as light-tight as possible on the skin surface and is lightly pressed against it. The closure with the skin prevents (or at least reduces) stray light from entering the measurement detector during the measurement process, thus leading to a poorer signal-to-noise ratio.

As explained above, the test arrangement comprises a measurement emitter ME in form a several lighting diodes, and a reference emitter RE in form a several lighting diodes. Those diodes for the reference and the measurement emitter can be controlled separately and are configured to emit light within the green, red and infrared spectrum. In front of the measurement emitter ME and the reference emitter RE, optics O1 are arranged, respectively, which have an aperture and a downstream focusing optics. The optics O1 are designed to project a substantially common focal point on the skin surface 6 in an area SE that can be illuminated by the measurement emitter and the reference emitter RE, respectively. A common focal point is not fundamentally required but is useful to avoid measurement differences due to irregularities at different measurement locations.

A measurement detector MD and a reference detector RD is arranged on the receiver side. These two detectors are also each preceded by a second optical system O2, which in turn comprises an aperture and one or more focusing lenses. The arrangement of the two detectors MD and RD and the upstream optics O2 is such that their respective detection point is located on the surface of the skin 6 in the area SD. The 3 detectors comprise one or more photodiodes, that are sensitive to the light emitted by the reference and measurement emitters.

Light emitted from this area thus strikes the detector MD or the detector RD, depending on the angle. The areas SE and SD are spatially slightly separated from each other, with the distance being in the range of a few millimetres. In addition, an optical barrier 4 is provided between the emitter side and the detector side within the detector arrangement, which extends from the window 7 into the detector arrangement 1A and is intended to prevent crosstalk of light from the measurement emitter or the reference emitter to the respective detectors.

The arrangement on the emitter side with the measurement emitter ME and the reference emitter RE is designed such that a measurement light beam generated by the measurement emitter is incident on the measurement area SE on the skin surface 6 at an angle α. The angle α is measured with respect to the skin surface or the surface of the window 7 placed on the skin. Thus the incident angle is given by 90°—α to the normal incidence. For the reference emitter RE the angle α is much large and close to 90°, thus the angle to the normal incidence is small in the range between 5° and 15°. Consequently, the reference emitter RE emits a reference light beam which falls on the area SE at a relatively steep angle above 80° and approximately at 90° (with respect to the skin surface). In other words, the reference emitter is positioned so that the light it generates is essentially perpendicular to the area SE to be illuminated.

Similarly, the measurement detector MD and the reference detector RD are also positioned at different angles to the normal incidence. The measurement detector MD is arranged so that scattered light from the area SD enters the measurement detector MD at a flat angle α′ in the range between 25° and 45°. In contrast, the reference detector RD detects mainly light that is backscattered substantially perpendicular to the surface of the skin and to the surface of the window 7. In a particular embodiment, the emitters and detectors are arranged symmetrically about an axis through the optical barrier, which in turn lie along a normal to the window and skin surface, respectively. This means that the measurement emitter and the measurement detector each have the same angle to the normal, as do the reference emitter and the reference detector.

Since the light beams penetrate the sample and the substance to be measured may be located within the sample and not at the surface, such an approach seems appropriate. In fact, for glucose measurements on body parts, it has been found that a certain distance between the SE and SD areas improves the measurement result. This effect can be explained on the one hand by the fact that light components reflected at the surface are effectively suppressed, so that the signal/noise ratio is improved.

The emitted light from Emitters ME and RE penetrates into the area SE and interacts there with the blood in the manner described here. The light falling into the detectors has thus propagated through the tissue and the probability of interaction with blood is greatly increased.

The test equipment according to FIG. 3 is used to perform SPO2 measurement but can also be used for the oral glucose tolerance test. The light emitted into the tissue comprises three main wavelength, namely 520 nm, 637 nm, and 940 nm, respectively. The signals are pulsed with a frequency between 20 Hz and 50 Hz and processed accordingly. The different light is emitted subsequently to each other.

The determination of the oxygen saturation referred to as SPO2 measurement is based on the ratio of PPG signals recorded at red and IR wavelengths, in the present case at 637 nm and 940 nm. Specifically, the ratio of perfusion indices at the two wavelengths is determined by illuminating tissue using short pulses of light at a sufficiently high repetition rate, in the present case 200 Hz, and subsequently sampling the corresponding photocurrent generated in the photodiode.

The raw photodiode current consists of an average signal level, referred to as DC level, and a modulation of the signal due to the beating of the heart, referred to as AC level. The AC signal can be estimated, for instance, by the difference between the peaks and the valleys of the raw signal. The perfusion index (PI) or relative modulation or modulation-percent is given by the ratio of AC to DC. FIG. 5 illustrates the two different signals over time. The relative modulation for red light is about 0.75%, as measured on the tip of a finger.

Likewise, the relative modulation for IR light is about 2%. To estimate the oxygen saturation, the ratio of the relative modulations is computed, referred to as the ratio of ratios; in this example the result is about 0.375. This value is assigned an oxygen concentration level by means of calibration with a reference device. In the embodiments it is assumed that 0.375 corresponds to nearly 100% of oxygen saturation for the present setup.

However, it is noted that in general, the ratio of ratios for a given oxygen saturation depends on optical design choices, in particular the relative separation between the light source and detector for each wavelength, the emission characteristics of each light source, the detection characteristics of the photo diode, and the performance of the optical barrier in between sources and detectors, as well as other system aspects that affects how light may propagate from a source to a detector. Hence, for a given piece of equipment, like the sensor described in the above Figures, one may calibrate the sensor prior to subsequent measurements.

FIG. 6 now illustrates the result of an oral glucose tolerance test (OGTT) under various angles of incidence and detection in regards to a reference glucose measurement. The reference level curve labelled with “reference” in FIG. 8 is established after not eating for several hours. The baseline level in this example is 88 mg/dl as established with an Abott Freestyle Libre 3® reference device. The reference device is readout once per minute during the OGTT.

At 9.58 in the morning, the test subject has a glucose containing food intake. These intakes can be different, but are usually containing lot of glucose to illustrate the effect. It is observed in previous measurements, particularly with regards to the reference measurement, that baseline fluctuation shortly after food intake can occur. To this extent, the drop in the reference between 10:00 and 10:15 may be a natural fluctuation of the reference device or a phenomenon caused by digesting the glucose containing food, e.g. the human body has to consume some available glucose in the blood first to obtain the digested glucose. About 15 minutes after food intake the reference glucose measurement starts to rise, with the peak of the glucose concentration being reached about 35 minutes after food intake with a maximum level of 128 mg/dl at 10:33. After peaking, the glucose concentration gradually decays over the next roughly 45 minutes to level out near the baseline at 86 mg/dl.

In addition to the reference measurement, a PPG measurement was conducted for 10 seconds with our bench-top using the test equipment illustrated in FIG. 4. The setup allows for different angular arrangement between the emitted incident light and the detected light. These are denoted E5° and E65° corresponding to said angular arrangement of the reference emitter RE (E5°) and the measurement emitter (E65°) with an angle of incidence as measured relative to the surface normal of 5° and 65°, respectively. The detectors RD and MD are referenced with D35° and D65°, respectively. Measurements were taken with a sampling rate of 200 Hz, where interleaved measurements are performed using the emitters ME und RE for the different wavelengths and pulse width <100 us for each of the illumination events. The light is detected with a photodiode at the location of detectors RD and ED.

As described above, all four permutations of reference and measurement channels are exercised for the available three wavelengths. This provides data over the range of emission and detection angles detailed in the description above.

The DC and AC components are then separated from the raw signal. FIG. 6 shows the AC coefficients of the raw signal in units of counts for the 940 nm (IR) wavelength together with the glucose reference measurements (the corresponding y-scale is on the right axis). The AC signal increases with increasing glucose concentrations for all channel permutations. However, the signal levels are different for the different angles, where higher emission angles produce greater AC signals.

In FIG. 7 the accompanying DC levels which indicate that the E65°/D65° channel combination produces about a 3× greater signal than the E5° channels. In other words, an incident light produces less DC signals if closer to the normal incidence value. The behaviour is seen not only at the infrared wavelength as depicted in FIG. 7, but also for other wavelength although here to a lesser degree. According to the scattering model, this behaviour may be due to the (forward-) scattering characteristic of tissue. The results presented in FIG. 7 also indicate that when integrating over an Lambertian emitter, higher angles with regard to the normal incidence (e.g. 90°−α>45°) result in a higher weighting for those angles.

The larger angles also indicate another phenomenon. It seems that with increasing glucose level in the blood (and a resulting decrease in the scattering), the DC value decreases as well and vice versa.

To obtain the perfusion index for the respective measurements at different angles and for a wavelength of 940 nm, one calculates the ration of the AC values/DC Values. The result is illustrated in FIG. 8 showing the perfusion index for each of the four angle combinations. For low incident angles, e.g. the E5° channels on RD, the correlation is slightly stronger than for higher incident angles. The AC component as seen in the previous pictus increases with increasing glucose content, as the DC portion becomes smaller. In comparison to the reference measurement, the sensitivity of such measurement for the E5° channels on RD is approximately factor 3, e.g. a glucose changes of 50% results in a change of the perfusion index obtained by the proposed method by a factor of 3. On the contrary for the combination of E65°/D65, corresponding to an angle of 65° to the normal incidence, the correlation is weakest.

In typical measurements systems, one can assume that the emitter and likewise the detector comprises a Lambertian radiation and detection characteristics. Hence, the angular measurements in the previous figures obtained during an OGTT can be used to approximately emulate such measurement system by summing up all the AC coefficients and all the DC coefficients, respectively, and then divide the sum of AC by the sum DC for red and IR wavelengths. The results are illustrated in FIG. 9.

The perfusion index for the red and infrared spectrum has a strong correlation to the glucose reference, which also has a potential impact on the SPO2 measurement. Hence, it is suitable to take the ratio of the two curves and determine whether the glucose correlation cancels out. At the beginning of the measurement, the perfusion index of the measurement using infrared light IR is about 50% greater than RED, whereas at the peak of the glucose test at about 10:35, the difference has shrunk to about 20%. Consequently the glucose correlation at different wavelength do not completely cancel out of the perfusion index and thus also not out of the ratio of ration used for the SPO2 measurement.

In other words, an SPO2 measurement using a Lambertian emission or detection characteristics at red and infrared light will be skewed if any glucose intake takes place during the measurement. This result may also hold for non-Lambertian characteristics or different wavelength. Hence, some form of compensation needs to be included in the determination of oxygen saturation. These findings are also evident n view of FIG. 10.

The Figure illustrates the ratio of ratios of perfusion indices PI_RED/PI_IR. While prior to the glucose intake and the oral glucose tolerance test, the ratio of ratios is around 0.6. However, after food intake, the ratio of ratios increases to a value of about 0.95, which is much closer to the value of ~1. Such change of ratio in a SPO2 measurement would usually indicate a significant drop in oxygen level. In view of the measurement about 35 minutes after food intake, the SPO 2 measurement based on this bench-top configuration would indicate an oxygen level that is too low, albeit the oxygen saturation was neither changed by external means, e.g., by increasing the pressure altitude of the test-environment or reducing the oxygen concentration of the ambient air, nor by internal mean, i.e. different breathing.

FIG. 11 illustrate a possible reason. The figure shows the ratio of ratios for different angle combinations. It seems that ratio of ratios PI_RED/PI_IR for the combination E65°/D65° and E65°/D35° settings exhibits strong Glucose signature in comparison to the other settings. Combined with the observation that the signal levels for higher angles outweigh the ones for the lower angles, the conclusion is that higher angles of incidence and detection exhibit a stronger correlation to glucose concentrations. They skew any SPO2 measurements if not taken into account or compensated. As a solution to this issue, it is proposed to limit the angle of illumination and detection to suitable ranges closer to normal incidence. Suitable ranges are between 0° and 35°.

This aspect is also supported by the result depicted in FIG. 12 showing the perfusion index for red light and infrared light at an emission angle of 65° (E 65 °) and detection angle of 65° (D65°) . The perfusion index for infrared light does not seem to respond as strong to a change in glucose concentration as the perfusion index of red light. As a solution, the arrangement for a SPO2 or any other measurement is configured to eliminate the higher angles for light in the infrared spectrum only. For example, an infrared VCSEL with narrow emission characteristic can be used and combined with a lighting diode in a single measurement system. Alternatively, VCSEL for red and infrared light can be used for SPO2 measurements, which both comprise a narrow emission characteristic. Likewise, as illustrated the embodiments above, the detector can use distributed Bragg gratings, suitable apertures at a distance above the PD, lenses, or other elements to limit the angle of detected light. A further alternative or in addition is to place collimating lenses over the LEDs.

In addition to the mechanical compensations, it is observed that green light is not sensitive to blood oxygen saturation. FIG. 13 shows the perfusion index for green light during the oral glucose tolerance test and observe a reasonably good correlation to the ratio of ratios of red and infrared perfusion indices. Consequently, on can use light of a third wavelength, which is not sensitive to blood oxygen but correlates with the blood glucose concentration to break and compensate the correlation for red and infrared wavelengths.

### LIST OF REFERENCES

2 finger
4 blocking barrier
5, 6 skin surface
7 window
10 substrate
11 emitter LED
13 detector, photodiode
14,15 emitter, LED
16 blocking barrier
20 detector, photodiode
30 control circuit
RE, ME emitter
RD, MD detector
O1, O2 optical elements
D1, D2 optical path
BC blood cell
S substrate
SE, SD area

1.-15. (canceled)

16. A method for determining a substance concentration in a sample comprising liquid containing particles, in particular oxygen in blood, wherein a refractive index of the liquid is dependent on a concentration of the substance dissolved therein and a density of particles in the liquid is substantially constant, comprising the steps of:
emitting a first light beam with a first wavelength onto the sample containing the liquid and detecting a first light component of the first light beam scattered by the sample;
emitting a second light beam with a second wavelength onto the sample containing the liquid and detecting a second light component of the second light beam scattered by the sample;
obtaining a ratio of the perfusion indices of the first light component and the second light component
wherein the step of obtaining comprises the step of:
adjusting the perfusion indices of at least one of the first light component and the second light component by a factor derived from at least one of:
a third light component of a third light beam with a third wavelength scattered by the sample in response to the third light beam emitted onto the sample;
a perfusion index obtained by a third light component of a third light beam with a third wavelength scattered by the sample in response to the third light beam emitted onto the sample;
emitting at least one of the first and second light beam and/or detecting at least one of the first and second light component in the range between 0° and 45° and in particular between 0° and 35° and in particular less than 30° of the normal incidence;
deriving the substance concentration or a change in the substance concentration from the adjusted perfusion indices;
wherein scattering of the third light beam with the third wavelength is substantially insensitive to a change of concentration of the substance dissolved in the liquid.

17. Method according to claim 16, wherein the liquid containing particles comprises a component, in particular glucose that influences the refractive index of the liquid.

18. Method according to claim 16, wherein the scattering of the first light beam, the second light beam and the third light beam is dependent on a concentration of another substance dissolved in the liquid.

19. Method according to claim 16, wherein the first component, the second first component and the third first component are detected from substantially the same location on the sample.

20. A method for determining the oxygen saturation in blood, comprising the steps of:
obtaining a first perfusion index based on a reflected first component of an incident first light beam with a first wavelength;
obtaining a second perfusion index based on a on a reflected second component of an incident second light beam with a second wavelength;
obtaining a reflected third component of an incident third light beam with a third wavelength, wherein the third light beam is in a wavelength range that is less sensitive to oxygen saturation than the first and second light beams;
adjusting at least one of the first and second perfusion indices in response to the obtained third component or a value derived therefrom;
determining the oxygen saturation or a change thereof from the at least one adjusted perfusion index and the respective other perfusion index.

21. Method according to claim 16, wherein the third wavelength is in the range between 500 nm to 550 nm.

22. Method according to claim 16, wherein the first light beam is in the range between 580 nm and 650 nm.

23. Method according to claim 16, wherein the second light beam is in the infrared range with a wavelength larger than 670 nm.

24. Optoelectronic device for determining a substance concentration in a sample comprising a liquid containing particles, in particular oxygen saturation, comprising:
a housing with an exit window and an optional entrance window;
at least one emitter unit arranged below the exit window and adapted to emit from the exit window onto the sample a first light beam having a first wavelength and at least one second light beams having a second wavelength;
at least one photodetector arranged below the entrance window and adapted to detect a first component scattered by the sample in response to the first light beam and a second component scattered by the sample in response to the second light beam;
an evaluation unit which is coupled to the at least one photodetector and is configured to
determine a modulation depth of the respective first and second components; and
determine a substance concentration or a change in the substance concentration in the sample from the changes of a ratio of the first and the second modulation depths.

25. The optoelectronic device according to claim 24, wherein the emitter unit is further configured to emit from the exit window onto the sample a third light beam having a third wavelength, said third wavelength selected such that the third light beam is scattered by the sample substantially independent of the substance concentration; and optionally the at least one photodetector arranged below the entrance window is adapted to detect a third component scattered by the sample in response to the third light beam.

26. The optoelectronic device according to claim 24, wherein the third light beam is in the green range of the spectrum, particularly between 510 nm and 560 nm.

27. The optoelectronic device according to claim 24, wherein at least one of the emitting angle of one of the first, second and third light beam and the detecting angle of the first, second and third component is in the range between 0° and 35° in regards to the normal incidence of the sample.

28. The optoelectronic device according to claim 24, further comprising at least one of:
an optical barrier arranged in the housing and extending from the exit window towards the housing bottom arranged between the emitter unit and the photodetector unit;
a DBR structure arranged on the at least one photodetector;
an aperture arranged on the at least one photodetector;
one or more collimating lenses arranged on at last one of the emitter unit and the at least one photodetector;
one or more VCSELs as part of the emitter unit.

29. The optoelectronic device according to claim 24, wherein the emitter unit comprises a ring-shaped form arranged around the at least one photodetector; or wherein the at least one emitter unit is arranged around the at least one photodetector.
